(19) United States
(12) Patent Application Publication
(10) Pub. No.: US 2008/0167277 A1
(43) Pub. Date: Jul. 10, 2008

(54) METHODS OF TREATING SKIN DISORDERS WITH CAFFEIC ACID ANALOGS

(76) Inventors: CHARLES CONRAD, Spring, TX (US); Waldemar Priebe, Houston, TX (US); Walter V. Klemp, Coldspring, TX (US); Robert A. Conrad, Spring, TX (US)

Correspondence Address: PATTERSON & SHERIDAN, L.L.P., Suite 1500, 3040 Post Oak Blvd., Houston, TX, 77056, US

(21) Appl. No.: 11/968,058

(22) Filed: Dec. 31, 2007

Related U.S. Application Data
(60) Provisional application No. 60/882,867, filed on Dec. 29, 2006.

(51) Int. Cl. A61K 31/59; A61K 31/216; A61K 31/26; A61P 17/06

(52) U.S. Cl. 514/168; 514/532; 514/528; 514/167

(57) ABSTRACT

Methods of treating skin diseases such as plaque psoriasis and inverse psoriasis include topical application of one or a combination of caffeic acid phenethyl ester, caffeic acid benzyl ester, and analogs thereof as an active agent. A pharmaceutical composition containing the active agent may further include a cell differentiating agent such as a retinoid, and/or vitamin D or analogs thereof. The method enables treatment of a lesion with active agent dosages of ten percent by weight, for example.

### CROSS-REFERENCE TO RELATED APPLICATIONS

This application claims benefit of U.S. provisional patent application Ser. No. 60/882,867, filed Dec. 29, 2006, which is herein incorporated by reference.

### BACKGROUND OF THE INVENTION

1. Field of the Invention

Embodiments of the invention generally relate to treating skin disease.

2. Description of the Related Art

Many different skin diseases or disorders exist. Some of them relate to persistent inflammation and abnormal cell growth. For example, psoriasis affects an estimated two to three percent of the world's population of which, plaque psoriasis (psoriasis vulgaris) comprises about 80 percent of these cases. Plaque psoriasis is characterized by raised, red inflamed lesions covered by a silvery white scale comprised of dead skin cells. Other forms of psoriasis include inverse psoriasis, pustular psoriasis, erythrodermic psoriasis, and guttate psoriasis. The lesions from psoriasis make the disorder both a medical and a cosmetic problem.

However, current treatments for psoriasis fail to produce satisfactory results. Prior treatments often take away some inflammation but do not cure or eliminate the disorder. Further, patients on such treatment may achieve only some improvement in their condition over long periods of several months.

Therefore, there exists a need for an improved method of treating skin disease such as plaque psoriasis and inverse psoriasis.

### SUMMARY OF THE INVENTION

Embodiments of the invention generally relate to methods of treating skin diseases such as plaque psoriasis and inverse psoriasis with a topical application of one or a combination of caffeic acid phenethyl ester (CAPE), caffeic acid benzyl ester, and analogs thereof as an active agent. A pharmaceutical composition containing the active agent may further include a cell differentiating agent such as a retinoid, and/or vitamin D or analogs thereof. The method enables treatment of a lesion with active agent dosages of ten percent by weight, for example.

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1 is a plaque psoriatic lesion on a patient prior to treatment, according to embodiments of the invention.

FIG. 2 is the plaque psoriatic lesion shown in FIG. 1 after the third day of the treatment, according to embodiments of the invention.

FIG. 3 is a plaque psoriatic lesion on a patient prior to treatment, according to embodiments of the invention.

FIG. 4 is the plaque psoriatic lesion shown in FIG. 3 after the fourth day of the treatment, according to embodiments of the invention.

FIG. 5 is the plaque psoriatic lesion shown in FIG. 3 after the thirteenth day of the treatment, according to embodiments of the invention.

FIG. 6 is a general structure showing analogs of caffeic acid phenylethyl ester.

FIG. 7 is a general structure showing analogs of caffeic acid benzyl ester.

### DETAILED DESCRIPTION

Embodiments of the invention generally relate to methods of treating skin diseases such as plaque psoriasis and inverse psoriasis with a topical application of a pharmaceutical composition.

According to one embodiment, the composition includes a caffeic acid phenethyl ester (CAPE). The composition in one embodiment includes a caffeic acid benzyl ester (CABE). The following depicts the structure of these two compounds:

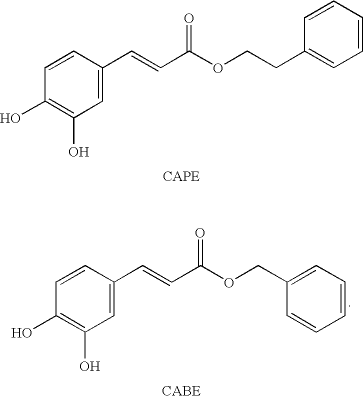

For some embodiments, analogs of caffeic acid esters as shown below may be employed in the composition:

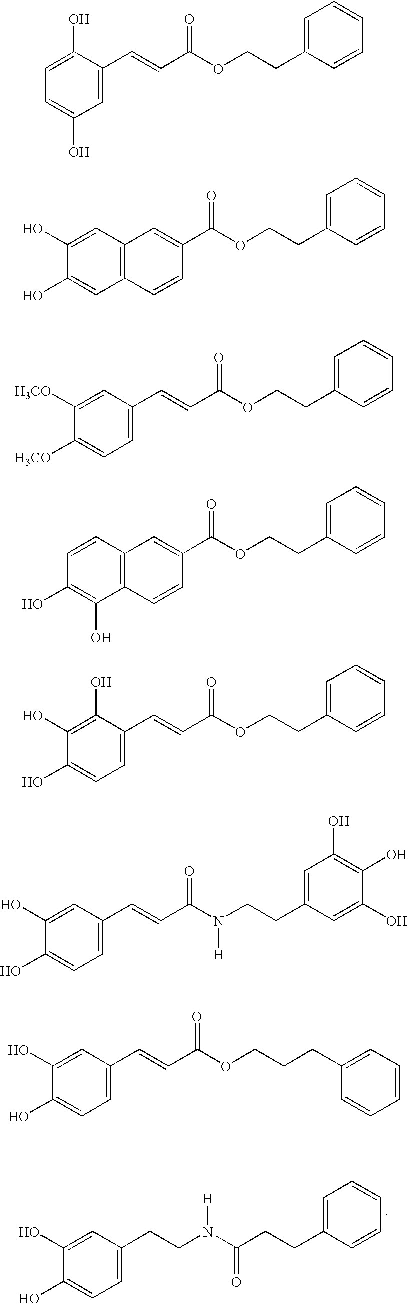

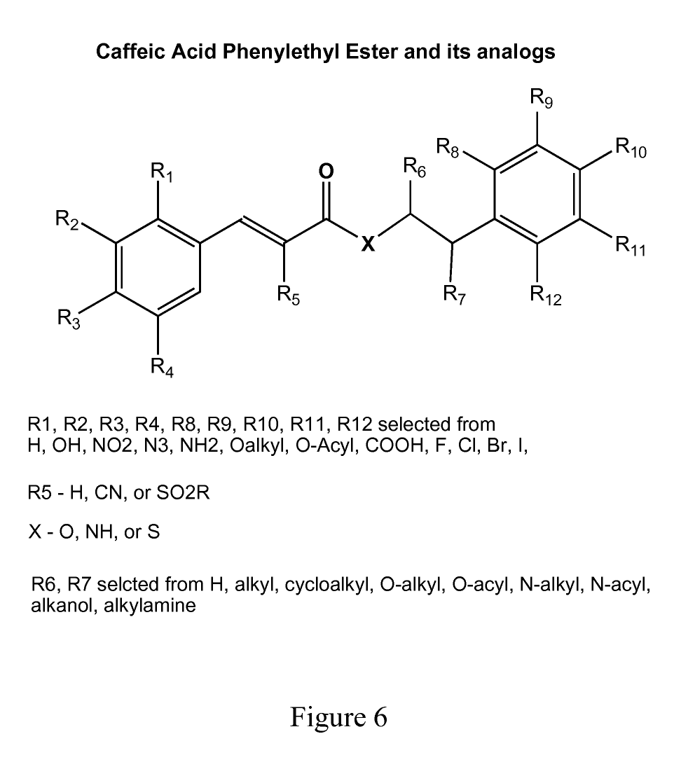

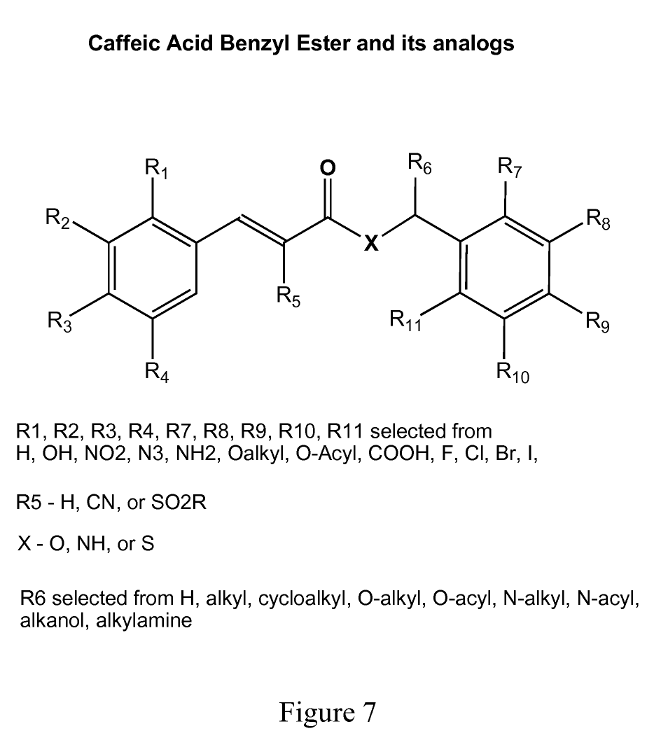

FIGS. 6 and 7 show general structures depicting analogs of caffeic acid phenylethyl ester and caffeic acid benzyl ester, respectively. The analogs include:

### Caffeic Acid Phenylethyl Ester and its Analogs

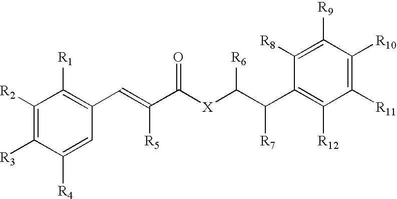 [0022] R1, R2, R3, R4, R8, R9, R10, R11, R12 selected from H, OH, NO2, N3, NH2, Oalkyl, O-Acyl, COOH, F, Cl, Br, I,
R5-H, CN, or SO2R
X—O, NH, or S
R6, R7 selected from H, alkyl, cycloalkyl, O-alkyl, O-acyl, N-alkyl, N-acyl, alkanol, alkylamine

### Caffeic Acid Benzyl Ester and its Analogs

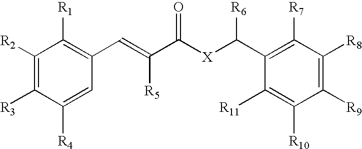 [0027] R1, R2, R3, R4, R7, R8, R9, R10, R11 selected from H, OH, NO2, N3, NH2, Oalkyl, O-Acyl, COOH, F, Cl, Br, I,
R5-H, CN, or SO2R
X—O, NH, or S
R6 selected from H, alkyl, cycloalkyl, O-alkyl, O-acyl, N-alkyl, N-acyl, alkanol, alkylamine
   and pharmaceutically acceptable salts thereof. For some embodiments, any of these analogs shown in FIGS. 6 and 7 of caffeic acid esters may be employed in the composition.

The term “alkyl” refers to saturated and unsaturated aliphatic groups including straight-chain, branched chain, alicyclic, and cyclic groups. Alkyl groups have 1 to 12 carbon atoms. In some embodiments, the alkyl groups may have 1 to 6 carbon atoms. In some embodiments, the alkyl groups may have 1 to 4 carbon atoms. The alkyl group may be optionally substituted with 1-3 substitutents selected from the group consisting of hydroxyl, alkylamine, —O-alkyl, acyl, —O-acyl, —NR-acyl, —C(O)—Oalkyl, —C(O)—NR-alkyl, thiol, and halo. R is H or alkyl. Suitable alkyl groups include methyl, isopropyl, —CH2-cyclohexyl, and cyclopropyl.

The term “SO2R” refers to the group —SO2-alkyl.

The term “alkanol” refers to the group -alkyl substituted with 1-3 hydroxy groups.

The term “alkylamine” refers to the group -alkyl substituted with 1-3 —NR2 groups, wherein each R group is independently selected from the group consisting of —H, and alkyl.

The term “acyl” refers to —C(O)R where R is alkyl.

The term “cyclic alkyl” or “cycloalkyl” refers to alkyl groups that are cyclic. Cycloalkyl groups have 1 to 12 carbon atoms. In some embodiments, the cycloalkyl groups may have 1 to 6 carbon atoms. In some embodiments, the cycloalkyl groups may have 1 to 4 carbon atoms. The cycloalkyl group may be optionally substituted with 1-3 substitutents selected from the group consisting of hydroxyl, alkylamine, —O-alkyl, acyl, —O-acyl, —NR-acyl, —C(O)—Oalkyl, —C(O)—NR-alkyl, thiol, and halo. R is H or alkyl. Suitable cyclic groups include cyclohexyl, cyclopentyl, and cyclopropyl.

The term “pharmaceutically acceptable salt” includes salts of the aforementioned compounds and prodrugs derived from the combination of a compound as set forth herein and an organic or inorganic acid or base. Suitable acids include HCl.

The following reaction illustrates synthesis of caffeic acid esters and its analogs.

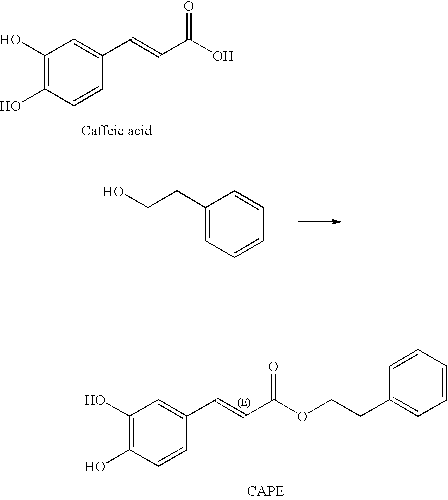

As shown, the general synthetic approach to CAPE and its analogs involves formation of an ester bond of caffeic acid or its analogs with phenyl derivatives containing a hydroxy group. Various methods of ester formation lead to formation of an ester bond between caffeic acid and its analogs and respective alcohols. For example, Fischer Esterification (Fischer-Speier Esterification) utilizes a Lewis or Brønstedt acid-catalyzed esterification of carboxylic acids with alcohols to give esters via a reaction in which the products and reactants are in equilibrium, as may be influenced by either removing one product from the reaction mixture (for example, removal of the water by azeotropic distillation or absorption by molecular sieves) or by employing an excess of one reactant. Alternative reactions employ coupling reagents such as dicyclohexylcarbodiimide (Steglich Esterification), preformed esters (transesterification), carboxylic acid chlorides or anhydrides. Esters may also be produced by oxidations, such as by the Baeyer-Villiger oxidation and oxidative esterifications. Similar procedures can be used to make the analogs.

Any one or a combination of the forgoing compounds represents suitable primary active agents for topical application to treat skin disorders or diseases. While not limited to any particular mechanism, it is believed that these primary active agents may inactivate one or more of six Signal Transducers and Activators of Transcription (STAT) pathways. For example, STAT3 inhibitors may remedy psoriasis disease states with complete effectiveness. STAT5 or its pathway or any other of the STAT1 through STAT6 and their pathways may also be blocked by the active agent to treat the skin. Further, interleukin 6 (IL-6) and interleukin 9 (IL-9) signaling may also be affected by the active agent during treatment.

The primary active agent may further be combined with a cell differentiating agent. Examples of cell differentiating agents include a retinoid, such as retinoic acid, vitamin D, vitamin D analogs, or a phorbol ester. The term vitamin D collectively refers to a group of structurally similar chemicals and their metabolites which include alfacalcidol (1-hydroxycholecalciferol), calcitriol (1,25-dihydroxycholecalciferol), cholecalciferol (vitamin D3), dihydrotachysterol (DHT) and ergocalciferol (vitamin D2). The active metabolite of vitamin D, 1,25-(OH)₂D₃, has a wide range of nonclassical actions in the body, such as regulation of cell growth and differentiation modulation of the immune system. This may be used in combinations with STAT3 inhibitors such as caffeic acid ester derivatives such as CAPE or CABE to be effective in the treatment of skin inflammatory and proliferation disorders such as psoriasis. The primary active agent may tend to stop an active lesion while the cell differentiating agent may lessen the likelihood of reactivation of the lesion to prevent recurrence.

Topical dosage forms include ointments, pastes, creams, lotions, gels, powders, solutions, sprays, and transdermal patches. The compounds described herein may be mixed with a carrier, which may include excipients, preservatives, or buffers. Exemplary carriers include petroleum jelly and dimethyl sulfoxide (DMSO). These dosage forms can also include excipients such as animal and vegetable fats, oils, waxes, paraffins, starch, tragacanth, cellulose derivatives, polyethylene glycols, silicones, bentonites, silicic acid, talc and zinc oxide, water or mixtures thereof.

In some embodiments, the topical dosage forms may contain 0.5 weight percent to 20.0 weight percent of the compounds described herein. For example, it has been found that 10.0 weight percent of CAPE in petroleum jelly is effective for treating psoriasis. The weight percent of the compounds described herein within the topical dosage forms may range from 1.0 weight percent to 15.0 weight percent or 5.0 weight percent to 11.0 weight percent. Treatment regimes with the topical dosage forms may occur daily, twice daily, three times daily or four times daily for durations, for example, of three weeks or four weeks or until symptoms are no longer present.

### EXAMPLE 1

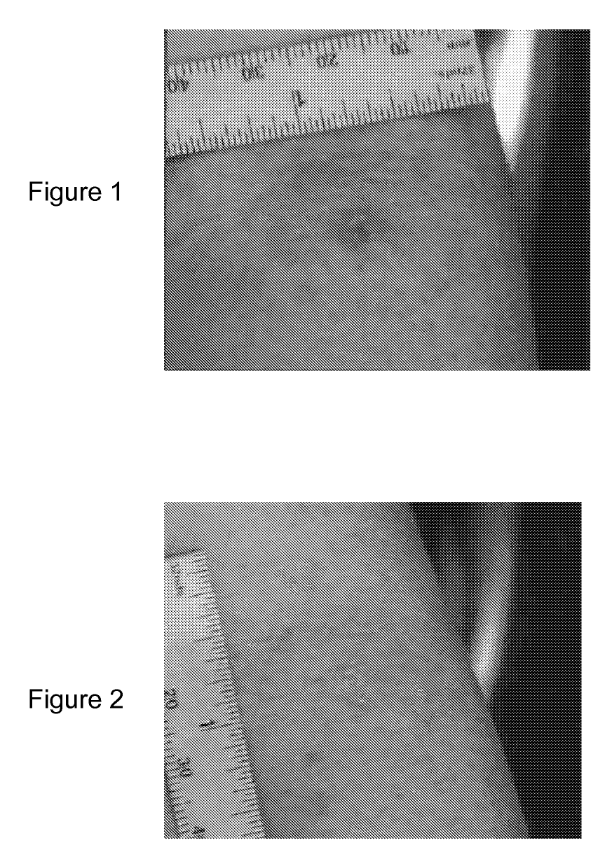

A solution containing about 0.8 weight percent CAPE solubilized in dimethyl sulfoxide (DMSO) was prepared. This mixture provided Solution 1 that was applied topically to three human patients three times daily. FIGS. 1-5 demonstrate ability of one embodiment of the invention to obtain complete resolution of psoriatic lesions.

FIG. 1 shows a plaque psoriatic lesion on a patient prior to any treatment. The patient applied the Solution 1 topically to the lesion three times daily throughout the treatment. FIG. 2 illustrates the plaque psoriatic lesion shown in FIG. 1 after the third day of the treatment.

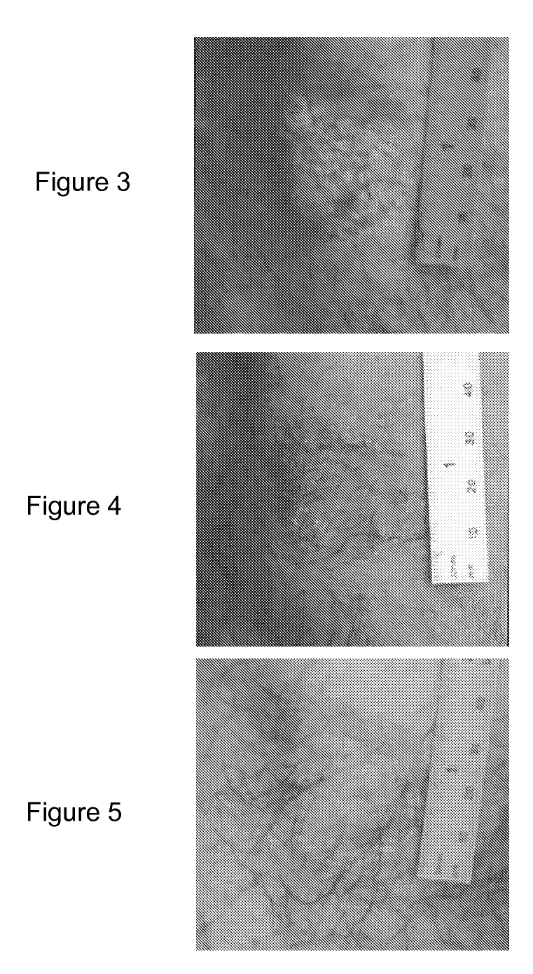

FIG. 3 shows a plaque psoriatic lesion on another patient prior to treatment with the Solution 1 topically applied three times a day to the lesion. FIG. 4 illustrates the plaque psoriatic lesion shown in FIG. 3 after the fourth day of the treatment. By the fourth day, visible improvement included reduced redness and scaliness of an affected area. FIG. 5 shows the plaque psoriatic lesion shown in FIG. 3 after the thirteenth day of the treatment. In about two weeks, thickness of the affected area returned to normal. As visible from the results, the treatment cured the psoriasis and not just reduced symptoms such as inflammation.

The description heretofore relates to applications for psoriasis. However, the invention may be utilized for other skin disorders and may benefit from agents described herein that inhibit cell proliferation. While the foregoing is directed to embodiments of the present invention, other and further embodiments of the invention may be devised without departing from the basic scope thereof, and the scope thereof is determined by the claims that follow.

What we claim is:

1. A method of treating plaque psoriasis or inverse psoriasis, comprising:
applying a topical pharmaceutical composition to a lesion, wherein the composition comprises one of caffeic acid phenethyl ester (CAPE), caffeic acid benzyl ester (CABE), an analog of CAPE, an analog of CABE, and a pharmaceutically acceptable salt thereof.

2. The method of claim 1, wherein the composition comprises a solubilizing agent selected from the group consisting of petroleum jelly and dimethyl sulfoxide (DMSO).

3. The method of claim 1, wherein the composition comprises caffeic acid benzyl ester.

4. The method of claim 1, wherein the composition further comprises a cell differentiating agent.

5. The method of claim 1, wherein the composition further comprises a retinoic acid as a cell differentiating agent.

6. The method of claim 1, wherein the composition further comprises vitamin D or a vitamin D analog as a cell differentiating agent.

7. The method of claim 1, wherein the composition comprises less than about 15.0 weight percent of the CAPE.

8. The method of claim 1, wherein the composition comprises less than about 15.0 weight percent of the CABE.

9. The method of claim 1, wherein applying the composition occurs multiple times daily until a lesion of the psoriasis disappears.

10. A composition for topical application to psoriatic lesions, comprising:
a carrier suitable for topical administration; and
one of caffeic acid phenethyl ester (CAPE), caffeic acid benzyl ester (CABE), an analog of CAPE, an analog of CABE, and a pharmaceutically acceptable salt thereof.

11. The composition of claim 10, wherein the composition comprises CAPE solubilized in one of petroleum jelly and dimethyl sulfoxide (DMSO).

12. The composition of claim 10, wherein the composition comprises caffeic acid benzyl ester.

13. The composition of claim 10, further comprising a cell differentiating agent.

14. The composition of claim 10, further comprising a cell differentiating agent selected from at least one of retinoic acid, vitamin D and a vitamin D analog.

15. The composition of claim 10, wherein the composition comprises less than about 15.0 weight percent of the CAPE.

16. The composition of claim 10, wherein the composition comprises 10.0 weight percent of the CAPE in a petroleum jelly.

17. The composition of claim 10, wherein the composition comprises less than about 15.0 weight percent of the CABE.

18. The composition of claim 10, wherein the composition comprises an effective amount of one of the CAPE, the CABE, the analog of CAPE, the analog of CABE, and the pharmaceutically acceptable salt thereof to inhibit a Signal Transducers and Activators of Transcription 3 (STAT3) signaling pathway.

19. A method of treating plaque psoriasis or inverse psoriasis, comprising:
inhibiting a Signal Transducers and Activators of Transcription 3 (STAT3) signaling pathway by applying a topical pharmaceutical composition to a lesion, wherein the composition comprises at least one of caffeic acid phenethyl ester (CAPE), caffeic acid benzyl ester (CABE), an analog of CAPE, an analog of CABE, and a pharmaceutically acceptable salt thereof in an effective amount to achieve inactivation of the STAT3 pathway.

20. The method of claim 19, wherein the composition comprises one of CAPE and CABE in the effective amount to achieve inactivation of the STAT3 pathway.
